(19) United States
(12) Patent Application Publication
(10) Pub. No.: US 2025/0049933 A1
(43) Pub. Date: Feb. 13, 2025

(54) DRUG DELIVERY SYSTEM FOR TARGETED CANCER TREATMENT

(71) Applicants: Misbahuddin Rafeeq, Jeddah (SA); Gaurav Gupta, Ajman (AE)

(72) Inventors: Misbahuddin Rafeeq, Jeddah (SA); Gaurav Gupta, Ajman (AE)

(21) Appl. No.: 18/922,478

(22) Filed: Oct. 22, 2024

(51) Int. Cl. A61K 47/59; A61K 9/51; A61P 35/00

(52) U.S. Cl. CPC A61K 47/593; A61K 9/5146; A61K 9/5192; A61P 35/00

(57) ABSTRACT

The present invention provides a drug delivery system for targeted cancer treatment, comprising: a GEM-PLGA complex, wherein the complex contains 150 mg of GEM-PLGA, equivalent to 25 mg of gemcitabine (GEM); Acetone in an amount of 10 ml as a solvent for the GEM-PLGA complex; Polyvinyl alcohol (PVA) solution in an amount of 20 ml, with a concentration of 2% w/v, used as a stabilizer for the formulation; and a Layer-by-Layer nanoparticle (LbL-NP) formulation encapsulating the GEM-PLGA complex and configured to release GEM in a controlled manner upon administration. The physiochemical properties of Caps-GEM LbL NPs was evaluated such as particle size, zeta potential, percent entrapment efficiency (% EE), loading of drug and In-vitro drug release behavior. The surface morphology of LbL NP's was evaluated by scanning electronic microscopy (SEM).

### FIELD OF THE INVENTION

The present invention generally relates to development and evaluation of gemcitabine and capsaicin loaded layer by layer nanoparticles for effective treatment of pancreatic cancer. In particularly, the present invention relates to composition for a drug delivery system for targeted cancer treatment.

### BACKGROUND OF THE INVENTION

With an estimated 85 percent death rate, pancreatic cancer is the fourth most common malignancy, necessitating the development of novel treatment modalities that would improve therapy results while having little or no side effects. Although, there has been significant advancement in the field of chemotherapy, surgery, and radiation therapy; the diagnosis of PCR remains very poor with 1 to 5 year, with total survival rate of almost 25% and 6% respectively. The present chemotherapy treatment do not provides more than one-year of survival benefits. PCR is more frequently observed in aged patients than in younger ones.

Smoking and family history are important concerns, despite the fact that the aetiology of PCR is complicated and varied. Unfortunately, only around 20% of individuals have localised, possibly treatable cancers, thus the vast majority of patients who arrive with PCR symptoms are already in advanced stages. Surgery is still the primary therapeutic option for individuals with resectable PCR. Adjuvant therapy is recommended for individuals who have a pancreatic resection with the goal of curing the disease. Chemotherapy is also the primary treatment for those who have advanced cancer.

GEM is a pyrimidine analogue with a low oral bioavailability (9%) due to a high first-pass metabolism rate. GEM is a water-soluble medication (log p=−1.4) having a half-life of 42 to 94 minutes for short infusions and 245 to 638 minutes for long infusions. Despite these drawbacks, it is the most widely used chemotherapy for pancreatic cancer, with a 6.7-month overall survival rate. The combination of Abraxane® (a human serum-bound paclitaxel nanoparticle formulation) with GEM, which was recently approved by the FDA, has only enhanced overall survival to 8.5 months.

Many researchers have found nanoparticle formulations to enhance GEM pharmacokinetics and pharmacodynamics because to its short half-life and hydrophilic nature.

Curcumin (CUR) is a low-toxicity hydrophobic polyphenol derived from the curcuma longa (turmeric) plant. CUR is well-known for its anti-inflammatory, anti-angiogenic, antioxidant, wound-healing, and cancer-fighting properties. CUR has been encapsulated into liposomes, polymers, or nanoparticles to enhance its water solubility, stability, and therefore bioavailability due to its water insolubility and instability. CUR has been given in conjunction with doxorubicin, paclitaxel, and docetaxel for cancer treatment.

Because of their tumor-targeted effectiveness, increased drug bioavailability, and optimal pharmacokinetics of hydrophilic and hydrophobic drugs, NP's formulations based on polymer conjugation with a drug have gotten a lot of interest in the field of cancer treatment.

Nanoparticle dosage forms in the form of LbL Nanoparticles are a new breakthrough in the field of nanoparticle dosage forms. Recent research has shown that LbL NPs have the potential to improve in-vivo pharmacokinetics, regulate the release of loading agents, and improve molecular-targeting capabilities.

In the view of the forgoing discussion, it is clearly portrayed that there is a need to develop and evaluate LbL Nps of GEM and Caps for targeted treatment of PC. This two-layered nanoparticle will show synergistic effect acts as follows a outer layer containing curcumin which has the anti-angiogensis and anti-fibrolytic effect that will shut down the blood vessel formation and loosen up the tight junction between the tumor cell. Whereas the nanoparticle containing the anticancer drug GEM will enter the tumor cell killing the pancreatic cancer cell.

### SUMMARY OF THE INVENTION

The present invention synthesis a GEM-PLGA complex utilising amino acids as a linker. Because amino acids have a rather weak connection, they may readily break under certain circumstances, releasing the GEM. Amino acid would act as a connection, and doing so may potentially alter the medication release pattern and provide more effective treatment outcomes. LbL NPs drug delivery systems may effectively combine several drug delivery methods and allow a higher degree of efficacy and lethality than is available using traditional methodologies. The particle size of the developed LbL NPs of GEM-Caps was found to be 79.94 nm. In order to get more drugs to stay in and permeate through cancerous tissue, particle size under 200 nm works best.

### BRIEF DESCRIPTION OF FIGURES

These and other features, aspects, and advantages of the present invention will become better understood when the following detailed description is read with reference to the accompanying drawings in which like characters represent like parts throughout the drawings, wherein:

FIG. 1: NMR spectrum of PLGA-GEM.

FIG. 2: FTIR spectra of GEM-PLGA conjugate.

FIG. 3: SEM photo of LbL NPs of GEM-Caps NPs.

FIG. 4: In-vitro drug release of GEM-Caps LbL NP's and individual drug NP's.

FIG. 5A: Effect of GEM-Caps LbL-NPs on pancreatic cells (BXPC-3 cells).

FIG. 5B: Cellular delivery of LBL NPs was visualised by flow cytometry and fluorescent imaging.

FIG. 6A: Effect of GEM-Caps LbL-NPs on pancreatic cells (BXPC-3 cells) for tumour development.

FIG. 6B: Effect of GEM-Caps LbL-NPs on pancreatic cells (BXPC-3 cells) for tumor inhibition rates.

FIG. 6C: Effect of GEM-Caps LbL-NPs on pancreatic cells (BXPC-3 cells) for body weight.

FIG. 7A: Distribution of GEM in 24 h tissues in vivo.

FIG. 7B: Distribution of Caps in 24 h tissues in vivo.

FIG. 8 illustrates a Table about Composition of GEM Caps LbL Np's.

FIG. 9 illustrates a Table about Characterization of GEM-Caps LbL NPs and individual NPs of GEM and Caps; and

FIG. 10 illustrates a Table about Stability observation of developed LbL NPs of GEM-Caps.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present invention will be described below in detail with reference to the accompanying drawings.

The present invention provides a composition for a drug delivery system, comprising 150 mg of a GEM-PLGA complex, wherein gemcitabine (GEM) is conjugated to poly (lactic-co-glycolic acid) (PLGA) using an amino acid linker; 10 ml of acetone, used as a solvent for the GEM-PLGA complex; and 20 ml of polyvinyl alcohol (PVA) solution, with a concentration of 2% w/v, acting as a stabilizing agent.

In an embodiment, the amino acid linker used in the GEM-PLGA conjugation is selected from the group consisting of glycine, alanine, and serine.

In another embodiment, the PVA solution is prepared in an aqueous medium to stabilize the nanoparticles during the encapsulation process.

In another embodiment, the GEM-PLGA complex is synthesized using a solvent evaporation method to form nanoparticles.

Further, the present invention provides a drug delivery system for targeted cancer treatment, wherein the drug delivery system further comprises a GEM-PLGA complex, wherein the complex contains 150 mg of GEM-PLGA, equivalent to 25 mg of gemcitabine (GEM); Acetone in an amount of 10 ml as a solvent for the GEM-PLGA complex; Polyvinyl alcohol (PVA) solution in an amount of 20 ml, with a concentration of 2% w/v, used as a stabilizer for the formulation; and a Layer-by-Layer nanoparticle (LbL-NP) formulation encapsulating the GEM-PLGA complex and configured to release GEM in a controlled manner upon administration;

In another embodiment, the GEM-PLGA complex is conjugated using an amino acid linker, enabling controlled release of gemcitabine (GEM) under physiological conditions.

In another embodiment, the Layer-by-Layer nanoparticle formulation is configured to have a particle size of less than 200 nm to enhance tumor penetration and retention.

In another embodiment, the PVA solution acts as a surfactant to maintain the stability of the nanoparticles during and after formulation.

In another embodiment, the GEM-PLGA complex utilizes succinic anhydride as a linker, which reacts with the hydroxyl groups of gemcitabine to form an ester bond, wherein the ester bond is designed to hydrolyze selectively in the acidic tumor microenvironment, triggering a controlled release of GEM specifically at the tumor site. The degradation of this linker ensures that GEM is not prematurely released during circulation, enhancing the targeting efficiency.

In another embodiment, the acetone used as a solvent for the GEM-PLGA complex is removed through a vacuum drying process, which is performed at a controlled temperature of 40° C. for 6 hours, wherein the duration and temperature are optimized to ensure complete removal of the solvent while preserving the structural integrity and bioactivity of the encapsulated GEM-PLGA nanoparticles, preventing aggregation and ensuring a homogenous particle size distribution.

In another embodiment, the Layer-by-Layer nanoparticle (LbL-NP) formulation incorporates a cationic polymer layer of polyethylenimine (PEI) during its assembly, wherein the PEI is applied in a molar ratio of 1:1 with the GEM-PLGA complex to enhance the electrostatic interactions between the nanoparticles and the negatively charged cell membranes of cancer cells. This facilitates higher uptake of the nanoparticles by cancer cells, improving the intracellular delivery of GEM and enhancing therapeutic efficacy.

In another embodiment, the biphasic release profile of the GEM-PLGA complex is controlled by using PLGA of two distinct molecular weights: 30 kDa for the initial phase of rapid GEM release and 50 kDa for the sustained release phase, wherein the variation in molecular weight modulates the degradation rate of the polymer matrix, allowing a precise control over the release kinetics, which maximizes the therapeutic window of GEM within the tumor microenvironment.

In another embodiment, a polyethylene glycol (PEG) coating is applied to the Layer-by-Layer nanoparticles to improve their colloidal stability and circulation time in the bloodstream, wherein the PEG coating, at a concentration of 5% w/v, prevents nanoparticle aggregation by steric stabilization and reduces clearance by the reticuloendothelial system.

In another embodiment, the lyophilization process for the GEM-PLGA nanoparticles is conducted by freezing the nanoparticles at −80° C., followed by drying under a vacuum pressure of 0.1 mbar for 48 hours.

In another embodiment, the GEM-PLGA nanoparticles are engineered to respond to enzymatic triggers in the tumor microenvironment by incorporating a matrix metalloproteinase-2 (MMP-2)-cleavable peptide linker, wherein the peptide linker is conjugated to the GEM-PLGA complex, allowing the nanoparticles to release GEM upon cleavage by MMP-2, an enzyme that is overexpressed in the tumor stroma.

In another embodiment, the PVA solution is modified by incorporating chitosan at a concentration of 0.5% w/v, which is added after the initial nanoparticle formation.

In another embodiment, the GEM-PLGA complex is synthesized by utilizing N-hydroxysuccinimide (NHS) as a coupling agent, facilitating the formation of amide bonds between GEM and PLGA to achieve a conjugation efficiency of over 90%.

In another embodiment, acetone is replaced by dichloromethane as a solvent during the GEM-PLGA conjugation process, with the solvent being removed under reduced pressure at 35° C. to minimize residual solvent content in the nanoparticles.

In another embodiment, the Layer-by-Layer nanoparticle formulation includes polycaprolactone (PCL) as an additional outer layer to enhance the mechanical stability and prolong the release of GEM over a period of 72 hours.

In another embodiment, polyethyleneimine (PEI) is added as a cationic component to the Layer-by-Layer assembly to improve cellular uptake and enhance transfection efficiency of the GEM-PLGA complex.

In another embodiment, trehalose is incorporated as a cryoprotectant during the lyophilization process to preserve the structural integrity of the nanoparticles upon reconstitution.

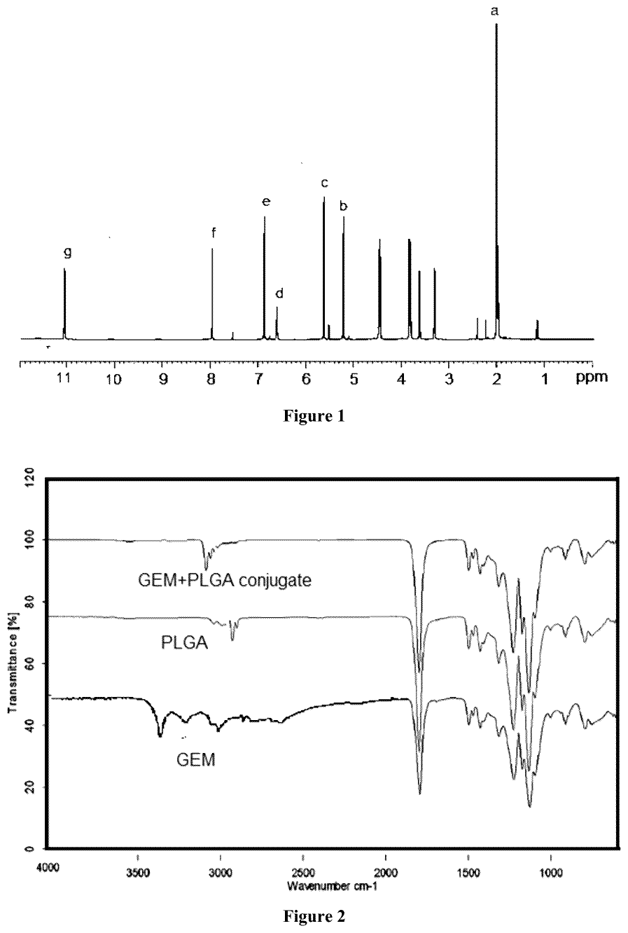

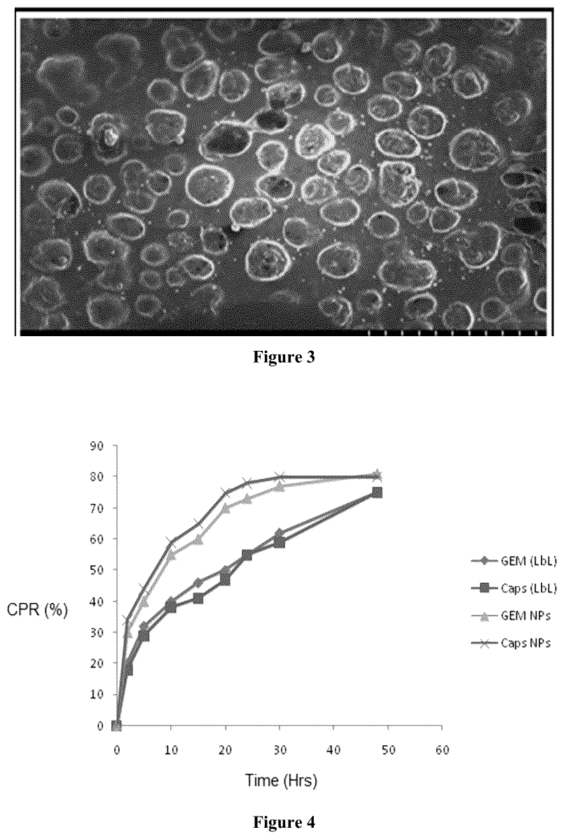

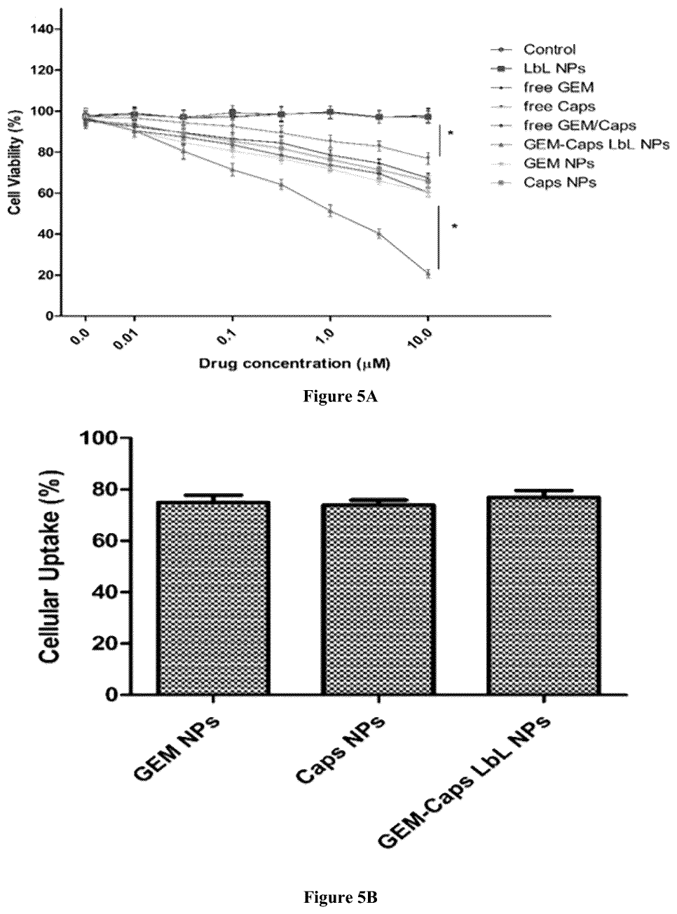

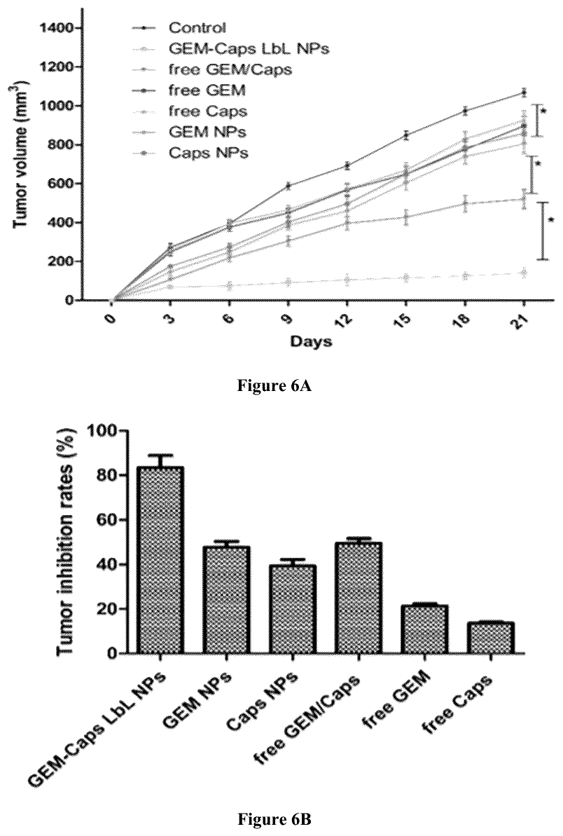

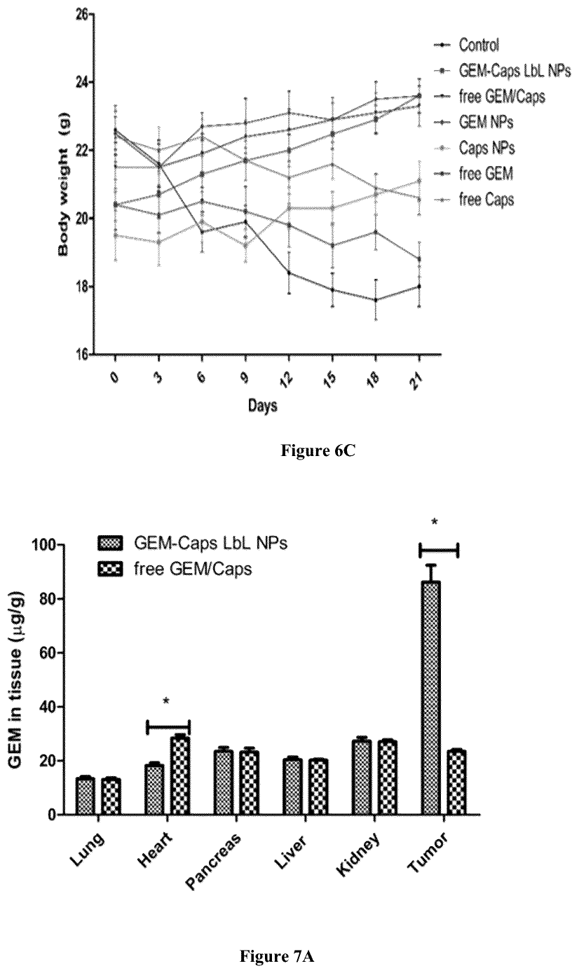

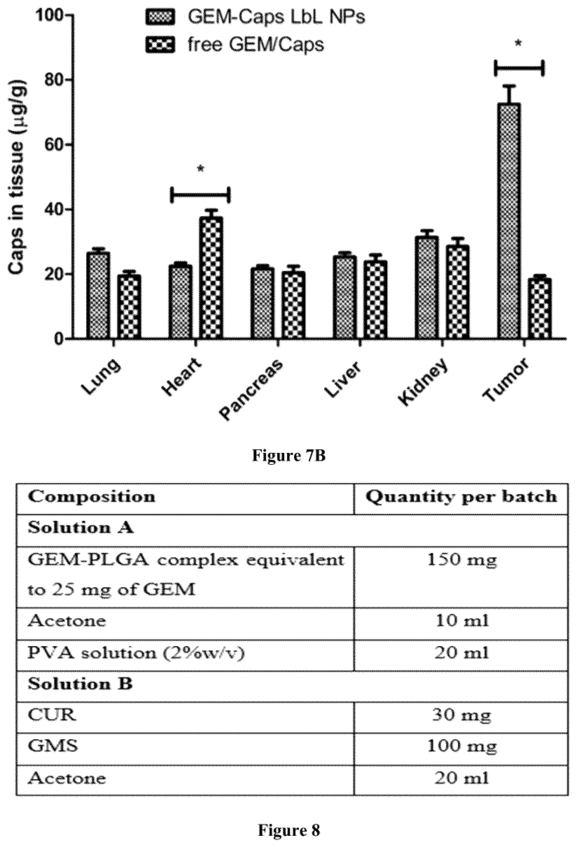

In another embodiment, the formulation incorporates chitosan nanoparticles at a concentration of 0.3% w/v to enhance the mucoadhesive properties of the system and promote prolonged retention at the tumor site.
FIG. 1: NMR spectrum of PLGA-GEM.
FIG. 2: FTIR spectra of GEM-PLGA conjugate.
FIG. 3: SEM photo of LbL NPs of GEM-Caps NPs.
FIG. 4: In-vitro drug release of GEM-Caps LbL NP's and individual drug NP's.
FIG. 5A: Effect of GEM-Caps LbL-NPs on pancreatic cells (BXPC-3 cells).
FIG. 5B: Cellular delivery of LBL NPs was visualised by flow cytometry and fluorescent imaging.
FIG. 6A: Effect of GEM-Caps LbL-NPs on pancreatic cells (BXPC-3 cells) for tumour development.
FIG. 6B: Effect of GEM-Caps LbL-NPs on pancreatic cells (BXPC-3 cells) for tumor inhibition rates.
FIG. 6C: Effect of GEM-Caps LbL-NPs on pancreatic cells (BXPC-3 cells) for body weight.
FIG. 7A: Distribution of GEM in 24 h tissues in vivo. And FIG. 7B: Distribution of Caps in 24 h tissues in vivo. 24 hours after intravenous treatment, mice were killed. The tissue from the tumour, kidneys, liver, pancreas, heart, and lungs was harvested, degraded in nitric acid, dried, and redissolved in an water and acetonitrile (50/50, v/v) solution. The data are given as mean±standard deviation, n=6. * indicates a probability of less than 0.05.

### Synthesis of GEM-PLGA Conjugate

PLGA conjugated of GEM was prepared by amidation of the carboxyl groups of GEM with PLGA's amine groups. First GEM-(COOH)₂ was prepared by adding H₂O₂ to GEM (solubilized in DMSO) to get GEM-(OH)₂, after that succinic anhydride was added to GEM-(OH)₂ and stirred for 11 hours. SDA-HCl and NHD were then added to the GEM-(COOH)₂ solution, followed by PLGA-NH₂ and stirred for 24 hours to produce GEM-PLGA. The GEM-PLGA conjugates obtained were packed in a glass vials and stored at 2-8° C.

### Preparation of GEM-Caps LbL Nanoparticles

GEM-Caps LbL NPs were formulated by a solvent diffusion method. Briefly, GEM-PLGA (150 mg) was solubilized in 10 ml of acetone and added into 20 ml of PVA (2%, w/v in water) solution under continuous stirring (solution A). Separately, Caps (30 mg) and GMS (100 mg) were dissolved in acetone (20 ml) (solution B). Now solution B was added drop wise into the Solution A (GEM-PLGA solution) under constant stirring (Table in FIG. 8). The obtained mixture was stirred overnight at room temperature to remove organic solvent. The final solution was centrifuged at 16,000 rpm for 15 min and washed with purified water thrice.

To formulate only GEM nanoparticles (GEM NPs), GEM (25 mg) and PLGA (170 mg) was utilized to replace GEM-PLGA and Caps was removed from the formulation process.

To formulate only Caps nanoparticles (Caps NPs), Caps (30 mg) and GMS (100 mg) was used and GEM was removed from formulation process.

To formulate drug-free LbL NPs, PLGA was utilized to replace GEM-PLGA and Caps was removed from the preparation process.

### Evaluation of Prepared GEM-Caps LbL NPs

### Characterization of Prepared GEM-Caps LbL NPs

The ¹NMR and FT-IR analysis was performed to characterize the PLGA-GEM complex.

### Particle Size and Zeta Potential

The particle size and zeta potential of developed LbL NP's were determined by using the Zeta sizer (Malvern zetasizer nano ZS, UK). Sample quantity of 1 ml was taken and diluted to 10 ml with distilled water. The size determination was carried out in samples after 10 minutes of sonication

### Scanning Electron Microscopy (SEM)

The shape of prepared LbL NP's was determined by using SEM (JEOL-JSM7900F, Tokyo, Japan). A small sample quantity was kept on surface of small metal stubs by help of adhesive tape. The sample coating was done with gold by help of sputter coater before testing. The SEM analysis was done by applying fixed voltages.

### Percent Entrapment Efficiency (% EE)

The % EE of developed LbL NP's was determined by ultra sonicating the NP's dispersion at 15000 rpm for 40 minutes. The supernatant obtain was diluted with 10 ml methanol and drug concentration was determined spcectrophotmetrically (Shimadzu 1800, Japan) at 267 nm and 281 nm for GEM and Caps respectively.

### In Vitro Drug Release

The in vitro release of GEM-Caps LbL NPs was determined by using Dialysis technique. 1 ml of LbL NP's (12.5 mg of GEM 15 mg of CUR) was added to dialysis membrane with molecular weight of 10,000 to 14,000 Da. The dialysis membrane than tied by a thread and immersed in a container having 15 ml of dissolution medium (phosphate buffer pH 7.4). The container was previously placed on an electromagnetic stirrer maintained at 37±0.5° C. temperature and dissolution medium was continuously stirred at 100 rpm by electromagnetic bead. 2 ml of sample was withdrawn at given time interval for 48 hours and same amount of dissolution medium was used to replenish it at each time intervals. The samples were estimated spectrophotometrically at λ max 267 nm, 281 nm for GEM and Caps respectively

### Stability Studies

The optimized GEM-Caps LbL NPs was evaluated for stability studies packed in amber color glass vials. The NP's were paced at two temperatures i.e. 2-8° C. & 25° C./60% RH for a period of 3 months. Initial evaluation parameters like description, particle size, zeta potential, drug content and percent in-vitro drug release was again examined after 3 months of stability study for any significant change.

### In Vitro GEM-Caps LbL NPs Cytotoxicity

The MTT assay was used to analyse LBL NPs the in vitro cytotoxicity on BXPC 3 cells. BXPC-3 cells were planted in 96-well plates with 5,000 cells per well, and therapy was to start the next day. The plates were incubated for 24 hours at 37 degrees Celsius in a 5% CO2 incubator. After this, cells were cultured for 72 hours in varying quantities of free GEM/Caps combo, free Caps, free GEM, LbL NPs, Caps NPs, GEM NPs, and GEM-Caps LbL NPs. In order to get accurate results, MTT (5 mg/mL in PBS) reagent was applied to each well and allowed to incubate for another 4 hours at 37 degrees Celsius. The formazan crystals generated after incubation were solubilized in 200 μL of DMSO, and the solution was scanned in a spectrophotometer at 570 nm.

### GEM-Caps LbL NPs Cellular Uptake

Cou-6 was used as a fluorescent probe to determine the cellular uptake efficiency of LBL-NPs. Cou-6 containing LBL NPs were prepared by dissolved Cou-6 (0.5 mg) in acetone along with PLGA or GEM-PLGA. BXPC-3 cells (1×10⁶ cells/well) were seeded in 6-well culture plate and incubated at 37° C. for 24 h. GEM-Caps LbL NPs, GEM NPs, Caps NPs (200 μg/mL) were added to the wells and incubated for 1 h. The cellular uptake efficiency of LBL NPs was visualized using a fluorescent-inverted microscope. A flow cytometer (BD Biosciences, Franklin Lakes, NJ) was applied to quantitatively analyze cellular uptake of LBL NPs after the cells were washed three times with D-Hank's solution, collected and centrifuged at 1500 rpm for 5 min.

### In Vivo Anti-Tumor Efficacy of GEM-Caps LbL NPs

BXPC-3 cells (2×10⁶ cells/mouse) were subcutaneous implanted into the right flank region of the BALB/c nude mice and let the tumor volumes grow to about 50 mm3. The tumor volumes were calculated using formulation: V (mm3)=½×(length)×(width)2.42 Then, the mice were randomized into 8 groups (n=6) and (1) 0.9% saline as a control, (2) GEM-Caps LbL NPs, (3) GEM NPs, (4) Caps NPs, (5) free GEM, (6) free Caps, (7) free GEM and caps were intravenously injected on day 0, 4, 8, 12 and 16. The behaviors of mice were monitored every 12 h along with the tumor growth and tumor volumes were measured with a vernier caliper every 3 days. After 21 days of injection, mice were sacrificed by cervical decapitation and the final tumor weight and body weight of mice were recorded to calculate the anti-tumor activity.

### In Vivo Tissue Distribution of GEM-Caps LbL NPs

Tumor-bearing Nude BALB/c mice were split into two groups of six, and were randomly selected and (1) GEM-Caps LbL NPs, (2) free GEM/Caps were injected intravenously. At 24 hours following injection, mice were euthanized. Tumor tissue from various organs was taken, both after harvesting and upon death, and then the tissue was heated in nitric acid to help it dissolve. The solution was then dried and redissolved in water and acetone. In the “Characterization of GEM-Caps LbL NPs” section, this project looked at how GEM and Caps are distributed.

### Statistical Analysis

All the data were statistically analyzed by using Chi-square test with help of Statistical Package for the Social Sciences 18.0 version. For determination of association, P<0.05 was considered statistically significant.

### Results

### GEM-Caps LbL NPs Characterization

FIG. 1 depicts the INMR spectra of PLGA-GEM complex as per the chemical structure. The peak (a) 2.01 ppm (c) 5.73 ppm and (d) 6.67 ppm belongs to GEM structure and peaks (b) 5.31 ppm (c) 6.85 ppm and (f) 11.18 ppm are characterizing PLGA structure. Peak (f) 7.81 ppm is characterizing amide linkage of GEM with PLGA. IR spectrum (v/cm−1): peaks at 2933, 1687, and 1054 belong to GEM; PLGA have the peaks of 1763, 1427, and 1093. The presence of amide linkage was confirmed by peak at 1661 of GEM-Caps NP's IR spectrum (FIG. 2).

### Particle Size and Zeta Potential

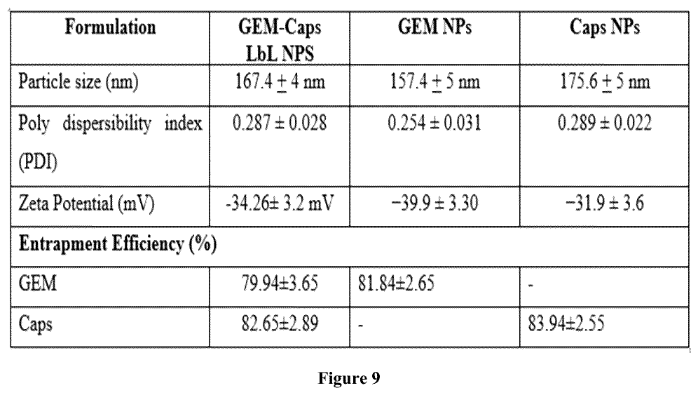

The particle size of LbL NPs of GEM-Caps was found to be 167.4±4 nm which similar in size of individual drug NPS (i.e. GEM NPs and Caps NPs). Hence, encapsulation of GEM and Caps in LbL NPS is not affecting the particle of individual drugs. The Zeta potential LbL Nps of GEM-Caps was observed negative −34.26±3.2 mV. The particle size and zeta potential data of developed GEM-Caps LbL NP's is given in Table in FIG. 9.

### Scanning Electron Microscopy (SEM)

SEM photographs were taken to assess the size and shape of developed GEM-caps LbL NP's. The surface morphology of LbL NP's revealed that prepared NP's were spherical in shape with smooth surface (FIG. 3). This confirmed that use method of LbL NP's preparation was able to successfully achieve uniformly distributed nanoparticles within nano size limit.

### Percent Entrapment Efficiency (% EE)

LbL NPs of GEM-Caps were evaluated for % EE. The developed LbL NPs formulation showed entrapment efficiency of 79.94%±3.65 for GEM and 82.65%±2.89 for Caps indicating the optimum amount of lipid is used for the formulation of LbL NP's (Table in FIG. 8).

### In Vitro Release of Drug

In-vitro drug release profile of GEM-Caps LbL NPs and individual NPS of GEM and Caps were studies. Although all the formulation showed sustained release pattern by LbL NPs of GEM-Caps appears to be more sustained release pattern (FIG. 4). There was no burst release was observed in all formulations. Nearly complete release from GEM-Caps LbL NPs and individual NPS of GEM and Caps was observed. The release of Caps was faster than GEM, which might be due to presence of CUR in the outer layer of LbL NP's so released easier than GEM.

### Stability Studies

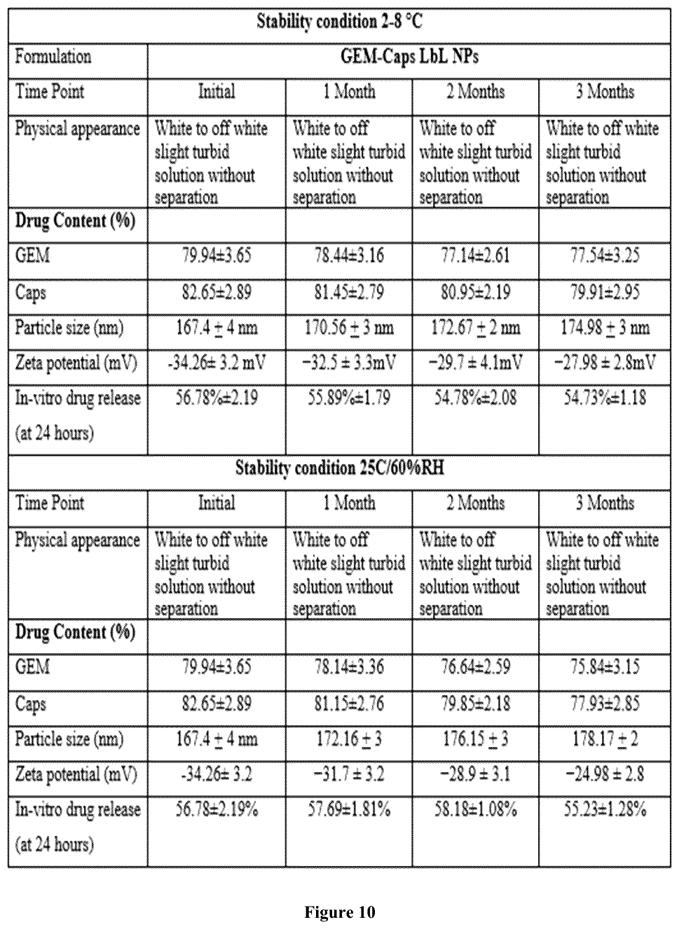

The developed LbL NP's of GEM-Caps were subjected for stabilities studies at 2-8°° C. (control) & 25° C./60% RH for a period of 3 months. No significant (p<0.05) was observed in physical parameters and drug release studies indicating stability of the formulation (Table in FIG. 10). Hence, it can be inferred that developed LbL NPs of GEM-Caps are stable at the 25° C./60% RH condition and could be stored for their self-life.

### In Vitro Cytotoxicity of GEM-Caps LbL NPs

The vitality of BXPC-3 cells is shown in FIG. 5A when exposed to GEM-Caps LbL NPs and other types of formulations. First, these cells didn't show cytotoxicity from blank LbL NPs, showing that the materials used to produce the NPs are safe. Cytotoxicity was seen for free medicines and the control group in BXPC-3 cells, but the medications were more effective than the control group but not as good as LbL NPs-loaded versions, which had a more profound influence on cancer cells (P<0.05). The most significant cytotoxicity was seen with GEM-Caps LbL NPs.

### GEM-Caps LbL NPs Cellular Uptake

Fluorescent pictures (FIG. 5B) and flow cytometry (FIG. 5C) were used to demonstrate the LbL NPs cellular absorption efficiency on BXPC-3 cells. Fluorescence microscopy images and quantitative findings showed that LbL NPs were taken up by cells at a rate of about 75%, demonstrating the carriers' strong uptake.

### In Vivo Anti-Tumor Efficacy of GEM-Caps LbL NPs

Tumor growth and body weight curves are presented in FIG. 6 after LbL-NPs were evaluated on pancreatic tumor-bearing mice in vivo. FIG. 6A shows that both GEM and Caps-soluble LBL NPs, as well as free medications, considerably halt tumour progression. (P<0.05). The most notable antitumor activity was found in GEM-Caps LbL NPs, which was significantly higher compared to other groups (P<0.05). The inhibition rates of tumor of GEM-Caps LbL NPs, and free GEM/Caps groups were 83.5±5.4 and 49.5±2.1% (FIG. 6B). FIG. 6C demonstrates that when mice were given medicines laden LbL NPs, their body weight did not alter substantially, but it did drop somewhat when mice were administered free drugs.

### In Vivo Tissue Distribution of GEM-Caps LbL NPs

The distribution of Caps or GEM in tissues varies in vivo (FIG. 7). Caps and GEM organization in tumor of GEM-Caps LbL NPs was greater than free GEM/Caps (P<0.05) (FIGS. 7A and B). However, the drug-encapsulated LbL NP group was notably more effective in decreasing GEM and Caps distribution in mouse hearts compared to the free drug group (P<0.05).

In the present invention, we synthesised a GEM-PLGA complex utilising amino acids as a linker. Because amino acids have a rather weak connection, they may readily break under certain circumstances, releasing the GEM. Amino acid would act as a connection, and doing so may potentially alter the medication release pattern and provide more effective treatment outcomes. LbL NPs drug delivery systems may effectively combine several drug delivery methods and allow a higher degree of efficacy and lethality than is available using traditional methodologies. The particle size of the developed LbL NPs of GEM-Caps was found to be 79.94 nm. In order to get more drugs to stay in and permeate through cancerous tissue, particle size under 200 nm works best.

Zeta potential is a critical physiochemical characteristic of NPs It changes the amount of cells that are affected by harmful effects as well as how well it is taken up by the cells. In case of positively charged NPs, cytotoxicity remains especially problematic, especially when the NPs are used in vivo. Positively charge NPs may cause shrinking of cells and decrease the number of cell mitosis. In order to omit these limitations, negatively charge NPs were developed in our study. The Zeta potential of developed GEM-Caps NPs was found to be −34.26 mV.

The surface morphology study of developed GEM-Caps LbL NPs revealed uniformly distributed and roughly spherical shaped NPs. The spherical NPs are less affected by shear and are able to interact more strongly with cell surfaces thereby increasing the cellular uptake.

The entrapment efficiency of both drug from developed GEM-Caps LbL NPs were found to be 79.94% for GEM and 82.65% for Caps which is comparable to when both drug entrapped individually in NPs i.e. 81.84% for GEM and 83.94% for Caps. Hence, it can have inferred from the study that entrapment of GEM and Caps together in one LbL NPs is not affecting their entrapment efficiencies. Sustained delivery of drugs from LbL NPs may decreases the systemic toxicity. Further, LbL NPs deliver time-dependent drugs, which could result in stronger cytotoxicity to the malignant cells.

In order to protect the formulation from undesirable effect of environment it becomes imperative to establish stability of the developed GEM-Caps LbL NPs. The developed GEM-Caps LbL NPs showed good stability at 2-8° C. and 25° C./60% RH conditions. There was no significant change observed in physical appearance, particle size, drug content and Zeta potential. Hence the developed formulations can be stored at 2-8° C. and 25° C./60 percent RH conditions for long period without any effect on physical appearance, particle size, drug content and Zeta potential.

The effectiveness of the method is dependent on how well nanoparticles are internalised and retained in cancer cells. Investigation into cellular uptake could yield evidence of how many LbL NPs got into the cells. A novel LbL NPs for the Cou-6 carrier were developed for imaging uptake. The LbL NPs were shown to have high capacity to penetrate cancer cells, as evidenced by the quantitative findings, which showed that almost 77% of the NPs were taken up by BXPC-3 cells. Due to the very efficient cellular absorption capacity, this system's therapeutic effectiveness may be improved by this feature.

The drug distribution and animal weight were studied for the GEM-Caps LbL NPs in vivo. Tumor tissue GEM-Caps LbL NPs were found to have greater GEM concentration than that of free GEM-Caps, and their levels were better than those in other experimental groups. Tumor EPR is the cause of this behaviour. NPs are concentrating in tumour tissues due to limited lymphatic outflow, which has been called the EPR effect. Mahmood et al reported a range of 172.6-479.65 nm in their investigation, and the present invention concurs since they obtained an EPR effect, which matches the range we recorded. A drug-encapsulated method that better demonstrates its ability target tumour tissue will be superior. GEM and CAPs levels in the heart were observed to be lower in the GEM-Caps LbL NP group, perhaps leading to less systemic toxicity when treating tumours. Nanoparticles should therefore be used in the medication delivery system, since it is both safe and essential. Using medicines delivered in LbL NPs at day 21, mice did not alter in weight, demonstrating that the systems were very well tolerated. The GEM-Caps LbL NPs may be used as a safe and effective therapy for pancreatic cancer due to the anticancer and tissue distribution tests conducted alongside the resultant findings.

GEM-PLGA conjugate was synthesized and encapsulated with Caps in LbL NPs formulation. GEM-Caps LbL NPs exhibited significantly high uptake and effective anti-tumor activity with no variation in the body weight. GEM-Caps LbL NPs is reported first time in combination for treatment of pancreatic cancer. The developed GEM-Caps LbL NPs is expected to be a novel and promising drug delivery system for synergistic treatment of pancreatic cancer. This developed LbL NPs delivery system can also be used as vector for co-encapsulation of different drugs and utilized for various cancer therapies.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible.

1. A drug delivery system for targeted cancer treatment, comprising:
a GEM-PLGA complex, wherein the complex contains 150 mg of GEM-PLGA, equivalent to 25 mg of gemcitabine (GEM);
Acetone in an amount of 10 ml as a solvent for the GEM-PLGA complex;
Polyvinyl alcohol (PVA) solution in an amount of 20 ml, with a concentration of 2% w/v, used as a stabilizer for the formulation; and
a Layer-by-Layer nanoparticle (LbL-NP) formulation encapsulating the GEM-PLGA complex and configured to release GEM in a controlled manner upon administration.

2. The drug delivery system of claim 1, wherein the GEM-PLGA complex is conjugated using an amino acid linker, enabling controlled release of gemcitabine (GEM) under physiological conditions.

3. The drug delivery system of claim 1, wherein the Layer-by-Layer nanoparticle formulation is configured to have a particle size of less than 200 nm to enhance tumor penetration and retention.

4. The drug delivery system of claim 1, wherein the PVA solution acts as a surfactant to maintain the stability of the nanoparticles during and after formulation.

5. The drug delivery system of claim 1, wherein the PVA solution is prepared in an aqueous medium to stabilize the nanoparticles during the encapsulation process.

6. The drug delivery system of claim 1, wherein the GEM-PLGA complex is synthesized using a solvent evaporation method to form nanoparticles.

7. The drug delivery system of claim 1, wherein the GEM-PLGA complex utilizes succinic anhydride as a linker, which reacts with the hydroxyl groups of gemcitabine to form an ester bond, wherein the ester bond is designed to hydrolyze selectively in the acidic tumor microenvironment, triggering a controlled release of GEM specifically at the tumor site. The degradation of this linker ensures that GEM is not prematurely released during circulation, enhancing the targeting efficiency.

8. The drug delivery system of claim 1, wherein the acetone used as a solvent for the GEM-PLGA complex is removed through a vacuum drying process, which is performed at a controlled temperature of 40° C. for 6 hours, wherein the duration and temperature are optimized to ensure complete removal of the solvent while preserving the structural integrity and bioactivity of the encapsulated GEM-PLGA nanoparticles, preventing aggregation and ensuring a homogenous particle size distribution.

9. The drug delivery system of claim 3, wherein the Layer-by-Layer nanoparticle (LbL-NP) formulation incorporates a cationic polymer layer of polyethylenimine (PEI) during its assembly, wherein the PEI is applied in a molar ratio of 1:1 with the GEM-PLGA complex to enhance the electrostatic interactions between the nanoparticles and the negatively charged cell membranes of cancer cells.

10. The drug delivery system of claim 1, wherein the biphasic release profile of the GEM-PLGA complex is controlled by using PLGA of two distinct molecular weights: 30 kDa for the initial phase of rapid GEM release and 50 kDa for the sustained release phase, wherein the variation in molecular weight modulates the degradation rate of the polymer matrix, allowing a precise control over the release kinetics, which maximizes the therapeutic window of GEM within the tumor microenvironment.

11. The drug delivery system of claim 1, wherein a polyethylene glycol (PEG) coating is applied to the Layer-by-Layer nanoparticles to improve their colloidal stability and circulation time in the bloodstream, wherein the PEG coating, at a concentration of 5% w/v, prevents nanoparticle aggregation by steric stabilization and reduces clearance by the reticuloendothelial system.

12. The drug delivery system of claim 1, wherein the lyophilization process for the GEM-PLGA nanoparticles is conducted by freezing the nanoparticles at −80° C., followed by drying under a vacuum pressure of 0.1 mbar for 48 hours.

13. The drug delivery system of claim 1, wherein the GEM-PLGA nanoparticles are engineered to respond to enzymatic triggers in the tumor microenvironment by incorporating a matrix metalloproteinase-2 (MMP-2)-cleavable peptide linker, wherein the peptide linker is conjugated to the GEM-PLGA complex, allowing the nanoparticles to release GEM upon cleavage by MMP-2, an enzyme that is overexpressed in the tumor stroma.

14. The drug delivery system of claim 1, wherein the PVA solution is modified by incorporating chitosan at a concentration of 0.5% w/v, which is added after the initial nanoparticle formation.

15. The drug delivery system of claim 1, wherein the GEM-PLGA complex is synthesized by utilizing N-hydroxysuccinimide (NHS) as a coupling agent, facilitating the formation of amide bonds between GEM and PLGA to achieve a conjugation efficiency of over 90%.

16. The drug delivery system of claim 1, wherein acetone is replaced by dichloromethane as a solvent during the GEM-PLGA conjugation process, with the solvent being removed under reduced pressure at 35° C. to minimize residual solvent content in the nanoparticles.

17. The drug delivery system of claim 1, wherein the Layer-by-Layer nanoparticle formulation includes polycaprolactone (PCL) as an additional outer layer to enhance the mechanical stability and prolong the release of GEM over a period of 72 hours.

18. The drug delivery system of claim 1, wherein polyethyleneimine (PEI) is added as a cationic component to the Layer-by-Layer assembly to improve cellular uptake and enhance transfection efficiency of the GEM-PLGA complex.

19. The drug delivery system of claim 1, wherein trehalose is incorporated as a cryoprotectant during the lyophilization process to preserve the structural integrity of the nanoparticles upon reconstitution.

20. The drug delivery system of claim 1, wherein the formulation incorporates chitosan nanoparticles at a concentration of 0.3% w/v to enhance the mucoadhesive properties of the system and promote prolonged retention at the tumor site.
